(19) United States
(12) Patent Application Publication
(10) Pub. No.: US 2002/0007835 A1
(43) Pub. Date: Jan. 24, 2002

(54) Method of treating opthalmalogical conditions using micro-acupuncture

(76) Inventor: Per Otte, Hot Springs Village, AR (US)

Correspondence Address: Boyd D. Cox, Suite 506, One McIlroy Plaza,, Fayetteville, AR, 72701, US

(21) Appl. No.: 09/794,816

(22) Filed: Feb. 26, 2001

Related U.S. Application Data
(60) Provisional application No. 60/184,872, filed on Feb. 25, 2000.

(51) Int. Cl. A61B 19/00

(52) U.S. Cl. 128/898

(57) ABSTRACT

A method of treating opthalmological conditions including macular degeneration, myopia, hyperopia, diabetic retinopathy and glaucoma in a patient, using microacupuncture comprises the invention. The method includes the steps of placing needles in the patient at key trigger points for a prescribed period of time and then removing the needles.

This application claims the benefit of U.S. Provisional Application No. 60/184,872, filed Feb. 25, 2000.

### BACKGROUND

The present invention is directed to a method of treating a patient suffering from one or more opthalmological conditions using micro-acupuncture. The opthalmological conditions include macular degeneration, myopia, hyperopia, diabetic retinopathy and glaucoma. The treatment comprises inserting needles in key trigger points on the patient's hands and feet for a prescribed period of time, then removing the needles. The key trigger points are specific to the condition(s) being treated. The micro-acupuncture treatment of the present invention significantly relieves the symptoms of macular degeneration, myopia, hyperopia, diabetic retinopathy and/or glaucoma.

### SUMMARY

The present invention is directed to a micro-acupuncture method for treating patients suffering from opthalmalogical conditions, including macular degeneration, myopia, hyperopia, diabetic retinopathy and/or glaucoma and to reduce the symptoms thereof.

The method of treatment comprises the steps of placing needles in the patient at the particular locations specified for the conditions, leaving the needles in place for at least approximately 20 minutes, then removing the needles from the patient. Subsequent treatments can be provided over the course of a specified time period, such as days or weeks.

It is an object of the present invention to provide a method for treating opthalmological conditions in a patient using micro-acupuncture.

It is a further object of the present invention to provide a method for treating macular degeneration in a patient.

It is a further object of the present invention to provide a method for treating myopia in a patient.

It is a further object of the present invention to provide a method for treating hyperopia in a patient.

It is a further object of the present invention to provide a method for treating diabetic retinopathy in a patient.

It is a further object of the present invention to provide a method for treating glaucoma in a patient.

It is a further object of the present invention to provide a method for significantly reducing the symptoms of macular degeneration in a patient.

It is a further object of the present invention to provide a method of acupuncture treatment for significantly reducing the symptoms of myopia in a patient.

It is a further object of the present invention to provide a method of acupuncture treatment that significantly reduces the symptoms of hypertopia in a patient.

It is a further object of the present invention to provide a method of acupuncture to treat diabetic retinopathy that significantly reduces the symptoms thereof in a patient.

It is a further object of the present invention to provide a method of acupuncture for treating glaucoma that significantly reduces the symptoms thereof in a patient.

It is a further object of the present invention to provide a method of acupuncture treatment that improves the vision of a patient.

It is a further object of the present invention to provide a micro-acupuncture method of treating opthalmalogical conditions including macular degeneration, myopia, hyperopia, diabetic retinopathy and glaucoma.

### BRIEF DESCRIPTION OF THE DRAWINGS

Reference is made to the accompanying drawings in which are shown illustrative embodiments of the invention and from which novel features and advantages will be apparent.

FIG. 1 is a schematic of a bottom view of a foot showing the key trigger points used in the preferred embodiments of the present invention.

FIG. 2 is a schematic of a patient's hands showing the key trigger points used in the preferred embodiments of the present invention.

FIG. 3 is a schematic of a patient's foot and hand having needles placed in the key trigger points for treating macular degeneration in a first preferred embodiment of the present invention.

FIG. 4 is a schematic of a patient's foot and hand having needles placed in the key trigger points for treating myopia and hyperopia in a second preferred embodiment of the present invention.

FIG. 5 is a schematic of a patient's foot and hand having needles placed in the key trigger points for treating diabetic retinopathy in a third preferred embodiment of the present invention.

FIG. 6 is a schematic of a patient's foot and hand having needles placed in the key trigger points for treating glaucoma in a fourth embodiment of the present invention.

### DETAILED DESCRIPTION

Referring now to FIG. 1, there is a shown a schematic diagram of a patient's foot 10 having five metatarsals, including a first metatarsal 11, a second metatarsal 12, a third metatarsal 13, a fourth metatarsal 14, and a fifth metatarsal 15. Each metatarsal 11-15 has a distal end and a proximal end. The distal ends are located closer to the digits 16 of the foot and the proximal ends are located closer to the heel 17 of the foot. Each metatarsal 11-15 is further distinguished as having an inner side and an outer side. The inner side of each metatarsal 11-15 is disposed closer to the edge of the foot aligned with the first metatarsal 11 and the outer side is disposed closer to the edge of the foot aligned with the fifth metatarsal 15.

The foot 10 has twelve key trigger points 20-31 as shown in FIG. 1. The key trigger points in the foot 10 are indicated by reference numerals as follows: KB at 20, BB at 21, GB at 22, BA at 23, GA at 24, KA at 25, STA at 26, LIA at 27, SPA at 28, LIB at 29, SPB at 30 and STB at 31.

Still referring to FIG. 1, the key trigger points of the foot have the following locations. The key trigger point KB 20 is located on a proximal end of the third metatarsal 13 at the bottom of the foot 10, while BB 21 is located on the outer side of the fifth metatarsal 15 at a proximal end thereof. GB 22 is located at an outer side near the proximal end of the fourth 14 metatarsal on the bottom of the foot 10. The point BA 23 is located at an outer side near the distal end of the fifth metatarsal 15. GA 24 is located at an outer side near the distal end of the fourth metatarsal 14 on the bottom of the foot 10. KA 25 is located approximate the middle of the distal end of the third metatarsal 13. STA 26 is positioned on the bottom of the foot at the outer side of the distal end of the second metatarsal 12. The key trigger point, LIA 27, is on the outer side of the first metatarsal 11 at a distal end thereof. SPA 28 is on the distal end of the first metatarsal 11 on the inner side thereof. Key trigger point, LIB 29, is on the proximal end of the first metatarsal 11 on an outer side thereof. The point, SPB 30, is positioned on the inner side of the proximal end of the first metatarsal 11, while STB 31 is located at the proximal end of the second metatarsal 12.

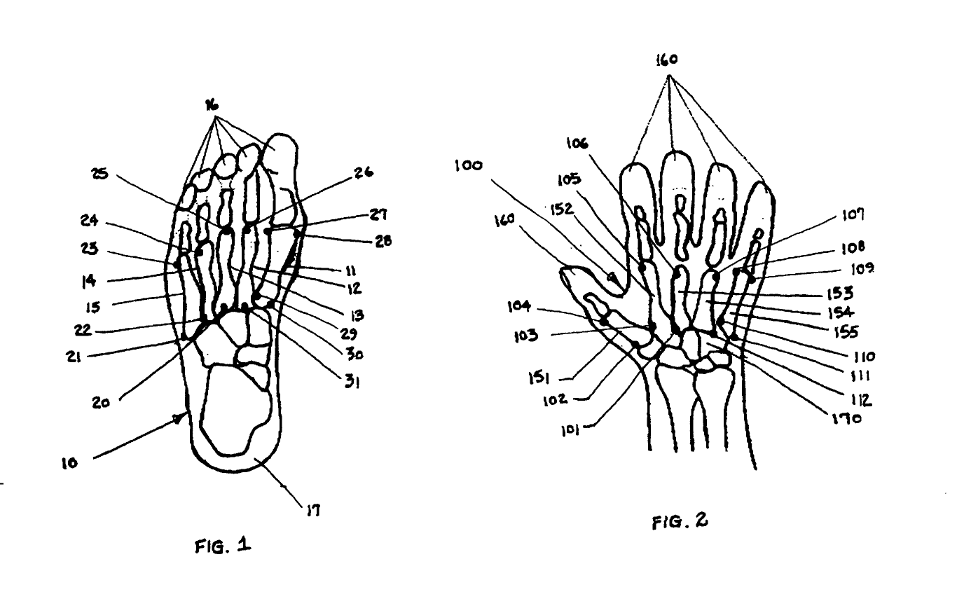

Referring now to FIG. 2, there is shown a schematic diagram of a patient's hand 100 having five metacarpals 151-155, including a first metacarpal 151, a second metacarpal 152, a third metacarpal 153, a fourth metacarpal 154, and a fifth metacarpal 155. Each metacarpal 151-155 has a distal end and a proximal end. The distal ends are disposed closer to the digits 160 of the hand and the proximal ends are disposed closer to the heel 170 of the hand. Each metacarpal 151-155 is further distinguished as having an inner side and an outer side. The inner side of each metacarpal 151-155 is disposed closer to the edge of the hand 100 aligned with the first metacarpal 151 and the outer side of each metacarpal 151-155 is disposed closer to the edge of the hand 100 aligned with the fifth metacarpal 155.

In the hand 100 as shown in FIG. 2, there are also twelve key trigger points 101-112 which are indicated by reference numerals as follows: CIRB at 101, LB at 102, CB at 103, LA at 104, CA at 105, CIRA at 106, EA at 107, HA at 108, TA at 109, HB at 110, TB at 111, and EB at 112.

The locations of the key trigger points on the hand 100 are as follows in FIG. 2. LA 104 is located adjacent an inner side of the hand 100, near the distal end of the first metacarpal 151. The key trigger point, EA 107, is approximate the distal end of the fourth metacarpal 154 adjacent the outer side thereof. HA 108 is located approximate the distal end of the fifth metacarpal 155 on an inner side thereof. The point, TA 109, is positioned approximate the distal end of the fifth metacarpal 155 on an outer side thereof.

It should be understood that the key trigger points of the foot 10 as shown in FIG. 1 are indicative of both the right and left feet of a patient. Although only the right foot is shown in the figures, the left foot and its key trigger points are mirrored images of the right foot. Likewise, the key trigger points of the right hand, as shown in FIG. 2, are also indicative of the key trigger points on the left hand with the left hand and its points being reversed to that of the right hand. Therefore, the foot and hand shown in FIGS. 1-6 are representational of both the left and right feet, and the left and right hands of a patient.

Reference is now made to FIG. 3 where the configuration of the needle placement is shown for a first embodiment of the invention which is a method for treating macular degeneration. In this first embodiment, needles 201-203 are placed in the left and right foot 10 at the respective key trigger points GA 24, STA 26, and LIA 27, respectively. Additional needles 204-206 are placed in the left and right hand 100 at the respective key trigger points EA 107, HA 108, TA 109.

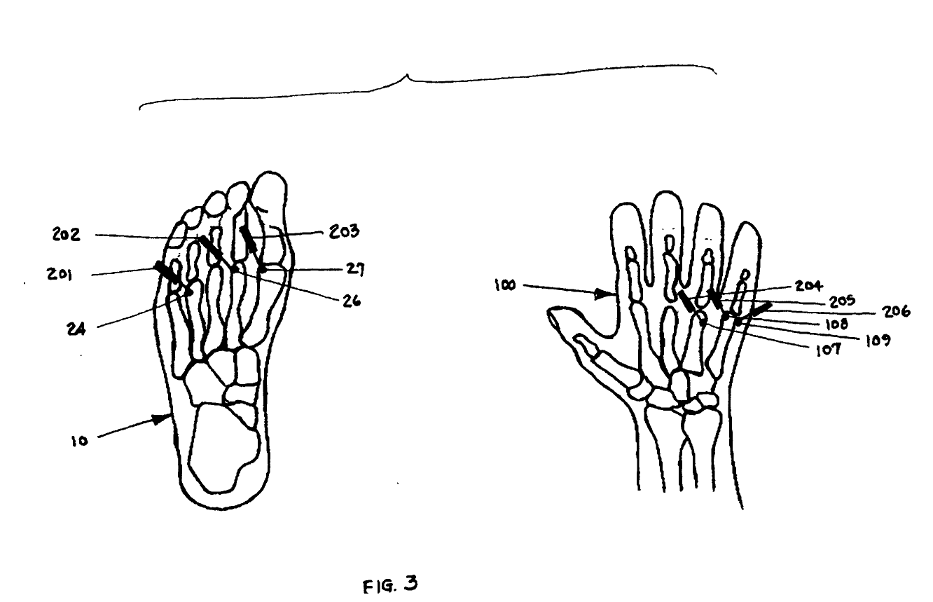

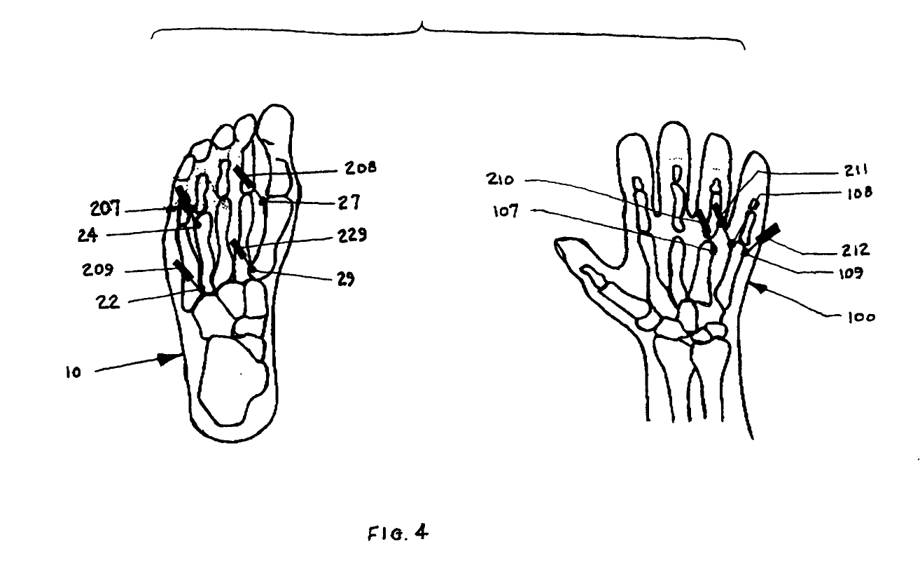

In a second embodiment of the present invention for treating myopia and hyperopia, the needle placement for the feet 10 and hands 100 is shown in FIG. 4. Needles 207-209, 229 are placed in the left and right foot 10 at the key trigger points GA 24, LIA 27, GB 22, and LIB 29, respectively. In the left and right hand 100, the needles 210-212 are placed at the respective key trigger points EA 107, HA 108 and TA 109, as indicated.

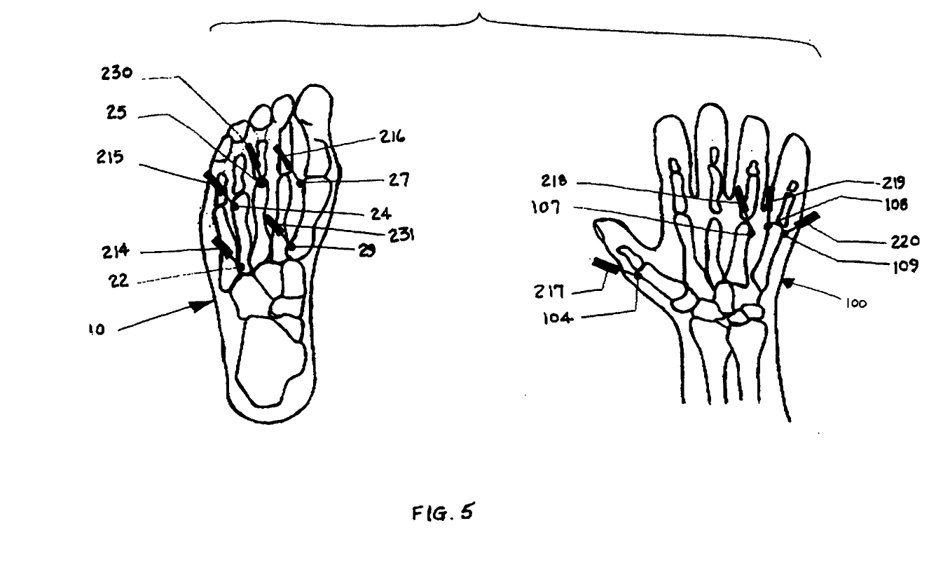

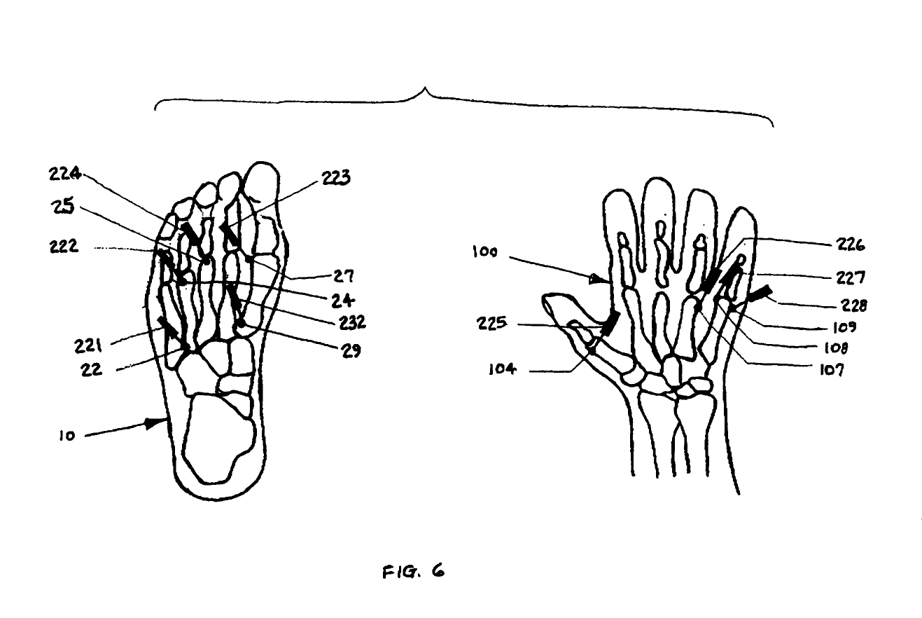

For treating diabetic retinopathy, the needle placement for a third embodiment of the present invention is shown in FIG. 5. The needles 214-216, 230, 231 are placed in the left and right foot 10 at the key trigger points GB 22, GA 24, LIA 27, KA 25 and LIB 29, respectively. In addition, needles 217-220 are placed in the left and right hand 100 at the respective key trigger points of LA 104, EA 107, HA 108 and TA 109.

In FIG. 6, the needle placement for a fourth embodiment for treating glaucoma, indicates needles 221-224, 232 placed in the left and right foot 10 at the key trigger points GB 22, GA 24, LIA 27, KA 25 and LIB 29 respectively. Additional needles 225-228 are placed in the left and right hand 100 at the key trigger points of LA 104, EA 107, HA 108 and TA 109.

In each of the four embodiments of the present invention, the patient undergoing treatment is preferably placed in a generally horizontal position with the feet and hands exposed. The needles are placed in the respective key trigger points of both feet 10 and hands 100 for the specified opthalmological condition. Preferably, these needles remain positioned at the respective key trigger points for the condition being treated for at least approximately 20 minutes. During the treatment with the needles placed in their respective positions, the patient remains relaxed in a generally horizontal position. After the elapsed time, the needles are removed from the patient.

Subsequent treatments with the needles inserted into their respective key trigger positions for the particular condition being treated can be administered, as needed. Each subsequent treatment comprises repeating the steps of the initial treatment after an amount of time has elapsed from the previous treatment. These additional treatments can be given over a course of time, such as days or weeks.

In the first embodiment of the present invention, the method for treating macular degeneration comprises the steps of placing needles 201-206 at the key trigger points in the feet and hands of a patient as shown in FIG. 3, waiting for a period of time, and then removing the needles 201-206.

The steps comprise inserting a needle 201 into the key trigger points GA 24 on the respective left and right foot 100; inserting the needle 202 into the key trigger points, STA 26 on the respective left and right foot 10; inserting a needle 203 into the key trigger points LIA 27 on the respective left and right foot 10; inserting a needle 204 into the key trigger points at EA 107 on the respective left and right hands 100; inserting a needle 205 into the key trigger points at HA 108 on the respective left and right hands 100; inserting a needle 206 into the key trigger points at TA 109 on the respective left and right hands 100 of the patient. After the needles have been inserted, the step of waiting for a period of time equal to about 20 minutes is performed. When the period of time has elapsed, the needles 201-206 are removed from the patient's feet and hands.

In the second preferred embodiment, the method for treating myopia and/or hyperopia comprises inserting the needles 207-212, 229 in the patient at the key trigger points in the hands and feet of the patient, as indicated in FIG. 4; waiting for a period of time; and removing the needles.

The steps of the method include inserting the needles 207-209, 229 into the respective key trigger points GA 24, LIA 27, GB 22, and LIB 29 of the patient's left and right feet 10; inserting the needles 210-212 into the respective key trigger points EA 107, HA 108, and TA 109 of the patient's left and right hands 100; waiting for a period of time of about 20 minutes; and removing all of the needles 207-212, 229 from the patient.

In the third embodiment, the method for treating retinopathy comprises inserting the needles 214-220, 230, 231 at the key trigger points in the feet and hands of a patient, as shown in FIG. 5, waiting for a period of time, and removing the needles from the patient.

Specifically, the method for treating retinopathy comprises the steps of inserting a needle 214 into the key trigger points GB 22 on the respective left and right foot 10; inserting a needle 215 into the key trigger points GA 24 on the respective left and right foot 10; inserting a needle 216 into the key trigger points LIA 27 on the respective left and right foot 10; inserting a needle 230 into the key trigger points KA 25 on the respective left and right foot 10; inserting a needle 231 into the key trigger points LIB 29 on the respective left and right foot 10; inserting a needle 217 into the key trigger points LA 104 on the respective left and right hand 100; inserting a needle 218 into the key trigger points EA 107 on the respective left and right hand 100; inserting a needle 219 into the key trigger points at HA 108 on the respective left and right hand 100; inserting a needle 220 into the key trigger points at TA 109 on the respective left and right hand 100; waiting for a period of time of approximately 20 minutes; and removing all of the needles 214-220, 230, 231 from the patient.

In the fourth embodiment, as shown in FIG. 6, the method for treating glaucoma comprises the steps of inserting a needle 221 into the key trigger points GB 22 on the respective left and right foot 10 of a patient; inserting a needle 222 into the key trigger points GA 24 on the respective left and right foot 10; inserting a needle 223 into the key trigger points LIA 27 on the respective left and right foot 10; inserting a needle 224 into the key trigger points KA 25 on the respective left and right foot 10; inserting a needle 232 into the key trigger points LIB 29 on the respective left and right foot 10; inserting a needle 225 into the key trigger points LA 104 on the respective left and right hand 100; inserting a needle 226 into the key trigger points EA 107 on the respective left and right hand 100; inserting a needle 227 into the key trigger points HA 108 on the respective left and right hand 100; inserting a needle 228 into the key trigger points TA 109 on the respective left and right hand 100; waiting for a period of time of approximately 20 minutes; and removing all of the needles 221-228, 232 from their respective key trigger points on the feet 100 and hands 10.

In each of the four embodiments disclosed herein, the method can include a series of treatments provided for each patient. Subsequent treatments in the series would comprise waiting an amount of time, and repeating the steps which comprise inserting the needles into the key trigger points of the patient as specified in the particular embodiment, waiting for a period of time of approximately 20 minutes and removing the needles in the patient's hands and feet. The frequency of repeated treatments is generally over a time period of days or weeks.

It should also be appreciated that various needles and equivalents known in the art can be used to perform the treatments comprising the preferred embodiments of the present invention.

Although the present invention has been described in considerable detail with reference to certain preferred versions thereof, other versions are possible. Therefore, the spirit and scope of the appended claims should not be limited to the description of the preferred versions contained herein.

1. A method of treating an opthalmalogical condition in a patient comprising the steps of:
(1) inserting a plurality of needles in respective key trigger points on hands and feet of the patient;
(2) waiting a period of time; and
(3) removing the plurality of needles from the patient;
wherein said respective key trigger points include GA located adjacent an outer, distal end of a fourth metatarsal on a bottom side of the patient's respective left and right foot; STA located adjacent an outer, distal end of a second metatarsal on the bottom side of the patient's respective left and right foot; LIA located adjacent an outer, distal end of a first metatarsal on the bottom side of the patient's respective left and right foot; EA located adjacent an outer, distal end of a fourth metacarpal on a top side of the patient's respective left and right hand; HA located adjacent an inner, distal end of a fifth metacarpal on the top side of the patient's respective left and right hand; TA located adjacent an outer, distal end of the fifth metacarpal on the top side of the patient's respective left and right hand.

2. The method of claim 1, wherein the opthalmalogical condition comprises macular degeneration.

3. The method of claim 1, wherein said period of time comprises approximately 20 minutes.

4. The method of claim 1, further comprising the additional steps of:
waiting an amount of time; and
repeating steps (1)-(3).

5. The method of claim 4, wherein said amount of time comprises at least one day.

6. A method of treating an opthalmalogical condition in a patient comprising the steps of:
(1) inserting a plurality of needles in respective key trigger points on hands and feet of the patient;
(2) waiting a period of time; and
(3) removing the plurality of needles from the patient;
wherein said key trigger points on the patient comprise GA located adjacent an outer, distal end of a fourth metatarsal on a bottom side of the patient's respective left and right foot; LIA located adjacent an outer, distal end of a first metatarsal on the bottom side of the patient's respective left and right foot; GB located adjacent an outer, proximal end of a fourth metatarsal on the bottom side of the patient's left and right foot; LIB located adjacent an outer, proximal end of the first metatarsal on the bottom side of the patient's left and right foot; EA located adjacent an outer, distal end of a fourth metacarpal on a top side of the patient's respective left and right hand; HA located adjacent to an inner distal end of a fifth metacarpal on the top side of the patient's respective left and right hand; TA located adjacent an outer, distal end of the fifth metacarpal on the top side of the patient's respective left and right hand.

7. The method of claim 6, wherein the opthalmalogical condition comprises myopia.

8. The method of claim 6, wherein the opthalmalogical condition comprises hyperopia.

9. The method of claim 6, wherein said period of time comprises approximately 20 minutes.

10. The method of claim 6, further comprising the additional steps of:
waiting an amount of time; and
repeating steps (l)-(3).

11. The method of claim 10, wherein said amount of time comprises at least one day.

12. A method of treating an opthalmalogical condition in a patient comprising the steps of:
(1) inserting a plurality of needles in respective key trigger points on hands and feet of the patient;
(2) waiting a period of time; and
(3) removing the plurality of needles from the patient;
wherein said respective key trigger points comprise GB located adjacent an outer, proximal end of a fourth metatarsal on a bottom side of the patient's respective left and right foot; GA located adjacent an outer, distal end of a fourth metatarsal on the bottom side of the patient's respective left and right foot; LIA located adjacent an outer, distal end of a first metatarsal on the bottom side of the patient's respective left and right foot; KA located adjacent a middle of the distal end of a third metatarsal on the bottom side of the patient's respective left and right foot; LIB located adjacent an outer, proximal end of the first metatarsal on the bottom side of the patient's respective left and right foot; LA located adjacent an inner, distal end of a first metacarpal on a top side of the patient's left and right hand; EA located adjacent an outer, distal end of a fourth metacarpal on the top side of the patient's respective left and right hand; HA located adjacent an inner, distal end of a fifth metacarpal on the top side of the patient's respective left and right hand; TA located adjacent an outer, distal end of the fifth metacarpal on the top side of the patient's respective left and right hand.

13. The method of claim 12, wherein said opthalmalogical condition comprises diabetic retinopathy.

14. The method of claim 12, wherein said period of time comprises approximately 20 minutes.

15. The method of claim 12, further comprising the additional steps of:
waiting an amount of time; and
repeating steps (1)-(3).

16. The method of claim 15, wherein said amount of time comprises at least one day.

17. A method of treating an opthalmalogical condition in a patient comprising the steps of:
(1) inserting a plurality of needles in respective key trigger points on hands and feet of the patient;
(2) waiting a period of time; and
(3) removing the plurality of needles from the patient;
wherein said key trigger points on the patient comprise GB located adjacent an outer, proximal end of a fourth metatarsal on a bottom side of the patient's respective left and right foot; GA located adjacent an outer, distal end of the fourth metatarsal on the bottom side of the patient's respective left and right foot; KA located adjacent a middle of the distal end of a third metatarsal on the bottom side of the patient's respective left and right foot; LIA located adjacent an outer, distal end of a first metatarsal on the bottom side of the patient's respective left and right foot; LIB located adjacent an outer, proximal end of the first metatarsal on a bottom side of the patient's respective left and right foot; LA located adjacent an inner, distal end of the first metacarpal on a top side of the patient's respective left and right hand; EA located adjacent an outer, distal end of a fourth metacarpal on the top side of the patient's respective left and right hand; HA located adjacent an inner, distal end of a fifth metacarpal on the top side of the patient's respective left and right hand; TA located adjacent an outer, distal end of a the fifth metacarpal on the top side of the patient's respective left and right hand.

18. The method of claim 17, wherein said opthalmalogical condition comprises glaucoma.

19. The method of claim 17, wherein said period of time comprises approximately 20 minutes.

20. The method of claim 17, further comprising the additional steps of:
waiting an amount of time; and
repeating steps (1)-(3).

21. The method of claim 20, wherein the amount of time comprises at least one day.
